(19) United States
(12) Patent Application Publication
(10) Pub. No.: US 2026/0318895 A1
(43) Pub. Date: Oct. 15, 2026

(54) CONTEXT-AWARE CLOUD SEEDING FOR WATER CARBON SEQUESTRATION

(71) Applicant: International Business Machines Corporation, Armonk, NY (US)

(72) Inventors: Jagabondhu HAZRA, Bangalore (IN); Manikandan PADMANABAN, Chennai (IN); Sarbajit Kumar RAKSHIT, Kolkata (IN)

(21) Appl. No.: 19/179,231

(22) Filed: Apr. 15, 2025

(51) Int. Cl. G01N 33/18; G01N 21/17; G01N 21/25

(52) U.S. Cl. CPC G01N 33/1826; G01N 21/25; G01N 2021/1795

(57) ABSTRACT

In some implementations, a device may receive satellite observation data comprising optical and microwave spectral reflectance data for a body of water. The device may determine a salinity, a pH, and a water carbon sequestration potential of the body of water using the satellite observation data and a geospatial foundation model. The device may determine one or more hotspots of potential carbon sequestration in the body of water based on the salinity, the pH, the water carbon sequestration potential, a phytoplankton turbidity saturation profile, and a marine species oxygen threshold profile. The device may output, based on the salinity, the pH, and the water carbon sequestration potential, an indication of an optimal set of hotspots of the one or more hotspots and an optimal mixture of alkaline substances for cloud seeding for the optimal set of hotspots.

### BACKGROUND

This disclosure generally relates to ocean carbon sequestration and cloud seeding.

### SUMMARY

Some aspects described herein relate to a method. The method may include receiving satellite observation data comprising optical and microwave spectral reflectance data for a body of water. The method may include determining a salinity, a pH, and a water carbon sequestration potential of the body of water using the satellite observation data and a geospatial foundation model. The method may include determining one or more hotspots of potential carbon sequestration in the body of water based on the salinity, the pH, the water carbon sequestration potential, a phytoplankton turbidity saturation profile, and a marine species oxygen threshold profile. The method may include outputting, based on the salinity, the pH, and the water carbon sequestration potential, an indication of an optimal set of hotspots of the one or more hotspots and an optimal mixture of alkaline substances for cloud seeding for the optimal set of hotspots.

Some aspects described herein relate to a computer system. The computer system may include a processor set, one or more computer-readable storage media, and program instructions stored on the one or more computer-readable storage media to cause the processor set to perform operations. The operations may include receiving satellite observation data of ocean water. The operations may include using the satellite observation data to fine-tune geospatial foundation models to infer salinity, pH, and water carbon sequestration potential of the ocean water. The operations may include determining one or more hotspots of potential carbon sequestration in the ocean water based on the salinity, the pH, and the water carbon sequestration potential. The operations may include performing an environmentally-aware cost-benefit analysis to determine an optimal set of hotspots of the one or more hotspots and an optimal mixture of alkaline substances for cloud seeding. The operations may include outputting an indication of the optimal set of hotspots and the optimal mixture of alkaline substances for cloud seeding.

Some aspects described herein relate to a computer program product. The computer program product may include one or more computer-readable storage media and program instructions stored on the one or more computer-readable storage media to perform operations. The operations may include evaluating, by a geospatial modeling system, satellite-based optical and microwave spectral reflectance data to estimate water salinity, pH, and carbon sequestration potential in a given ocean region. The operations may include identifying, by the geospatial modeling system, hotspots of potential carbon sequestration in the ocean region based on the water salinity, the pH, and the carbon sequestration potential. The operations may include outputting, by the geospatial modeling system, an indication of an optimal set of hotspots of the hotspots and an optimal mixture of alkaline substances for cloud seeding based on enhancing ocean-based carbon sequestration and minimizing environmental harm.

### BRIEF DESCRIPTION OF THE DRAWINGS

FIG. 1 is a diagram of an example computing environment for context-aware water carbon sequestration.

FIG. 2 is a diagram of an example implementation associated with cloud seeding for carbon sequestration.

FIG. 3 is a diagram of an example implementation associated with context-aware water carbon sequestration.

FIG. 4 is an example of a spatial map.

FIG. 5 is a flowchart of an example process associated with context-aware cloud seeding for water carbon sequestration.

FIG. 6 is a flowchart of an example process associated with context-aware cloud seeding for water carbon sequestration.

FIG. 7 is a flowchart of an example process associated with context-aware cloud seeding for water carbon sequestration.

### DETAILED DESCRIPTION

The following detailed description of example implementations refers to the accompanying drawings. The same reference numbers in different drawings may identify the same or similar elements.

Ocean carbon sequestration has been recognized as a promising approach to reducing carbon dioxide in the atmosphere. One common method of carbon sequestration in oceans is through alkalinity enhancement, where dissolved carbon dioxide is converted into dissolved bicarbonates. However, if not performed properly, this approach can have negative impacts on marine ecosystems, particularly on coral reefs, phytoplankton, and other marine species.

Coral reefs, which are essential for marine biodiversity, can be harmed by increased alkalinity levels, which can exceed their bicarbonate saturation profiles. Phytoplankton, which are crucial for marine food webs and carbon cycling, can be impacted by increased turbidity levels, which can block sunlight and interfere with their photosynthetic activity. Additionally, the breakdown of added minerals can consume oxygen, leading to low oxygen levels, or hypoxia, which can create “dead zones” where most marine life struggles to survive. Current methods of ocean carbon sequestration often fail to consider such environmental constraints, which can lead to unintended consequences.

Some implementations described herein provide a computer system that uses a technically sophisticated approach to ocean carbon sequestration that takes into account the complex relationships between alkalinity levels, coral reefs, phytoplankton, and other marine species. The computer system may identify optimal locations (hotspots) for water carbon sequestrations and optimal amounts of alkaline substances for the cloud seeding to enhance the water carbon sequestration. For example, the computer system may receive satellite observation data, including optical and microwave spectral reflectance data, for a body of water, and use a geospatial foundation model to determine salinity, pH, and water carbon sequestration potential of the body of water. The computer system may then determine one or more hotspots of potential carbon sequestration in the body of water based on the salinity, pH, water carbon sequestration potential, a phytoplankton turbidity saturation profile, and a marine species oxygen threshold profile. The computer system may output, based on the salinity, pH, and water carbon sequestration potential, an indication of an optimal set of hotspots and an optimal mixture of alkaline substances for cloud seeding for the optimal set of hotspots.

In this way, the computer system identifies optimal locations and optimal amounts of alkaline substances for cloud seeding to enhance ocean carbon sequestration. For example, the computing device may determine spatial distributions of coral reefs, phytoplankton, and low oxygen zones in the body of water, and may perform an environmentally-aware cost-benefit analysis to determine the optimal set of hotspots and optimal mixture. The computer system may evaluate a tradeoff between an additional gain from carbon credits for sequestration and a cost of deploying the optimal mixture. The computer system may determine the optimal amount such that the optimal amount maximizes carbon sequestration without impacting a health of coral reefs, phytoplankton, and other marine species. As a result, the computer system may optimize its computational efficiency in processing satellite data, reduce data transmission overhead by strategically selecting data for transmission, and minimize computational resource utilization for the environmentally-aware cost-benefit analysis. In this way, the computer system may conserve processing resources, memory resources, network resources, and/or the like.

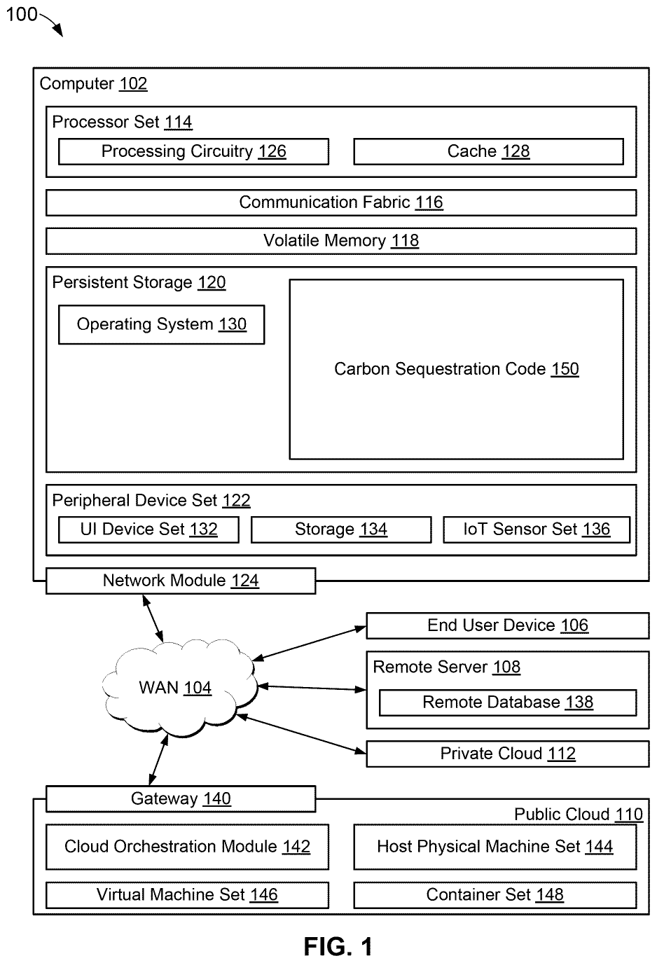

FIG. 1 is a diagram of an example computing environment 100 for context-aware water carbon sequestration, as described herein.

Computing environment 100 contains an example of an environment for the execution of at least some of the computer code involved in performing the inventive methods, such as carbon sequestration code 150. In addition to carbon sequestration code 150, computing environment 100 includes, for example, computer 102, wide area network (WAN) 104, end user device (EUD) 106, remote server 108, public cloud 110, and private cloud 112. In this embodiment, computer 102 includes processor set 114 (including processing circuitry 126 and cache 128), communication fabric 116, volatile memory 118, persistent storage 120 (including operating system 130 and carbon sequestration code 150, as identified above), peripheral device set 122 (including user interface (UI) device set 132, storage 134, and Internet of Things (IoT) sensor set 136), and network module 124. Remote server 108 includes remote database 138. Public cloud 110 includes gateway 140, cloud orchestration module 142, host physical machine set 144, virtual machine set 146, and container set 148.

Computer 102 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network, or querying a database, such as remote database 138. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 102, to keep the presentation as simple as possible. Computer 102 may be located in a cloud, even though it is not shown in a cloud in FIG. 1. On the other hand, computer 102 is not required to be in a cloud except to any extent as may be affirmatively indicated.

Processor set 114 includes one, or more, computer processors of any type now known or to be developed in the future. Processing circuitry 126 may be distributed over multiple packages (for example, multiple, coordinated integrated circuit chips). Processing circuitry 126 may implement multiple processor threads and/or multiple processor cores. Cache 128 is memory that is located in the processor chip package(s) and is typically used for data or code that should be available for rapid access by the threads or cores running on processor set 114. Cache memories are typically organized into multiple levels depending upon relative proximity to the processing circuitry. Alternatively, some, or all, of the cache for the processor set may be located “off chip.” In some computing environments, processor set 114 may be designed for working with qubits and performing quantum computing.

Computer-readable program instructions are typically loaded onto computer 102 to cause a series of operational steps to be performed by processor set 114 of computer 102 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 128 and the other storage media discussed below. The program instructions, and associated data, are accessed by processor set 114 to control and direct performance of the inventive methods. In computing environment 100, at least some of the instructions for performing the inventive methods may be stored in carbon sequestration code 150 in persistent storage 120.

Communication fabric 116 is the signal conduction path that allows the various components of computer 102 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 118 is any type of volatile memory now known or to be developed in the future. Examples include dynamic type random access memory (RAM) or static type RAM. Typically, volatile memory 118 is characterized by random access, but this is not required unless affirmatively indicated. In computer 102, the volatile memory 118 is located in a single package and is internal to computer 102, but, alternatively or additionally, the volatile memory may be distributed over multiple packages and/or located externally with respect to computer 102.

Persistent storage 120 is any form of non-volatile storage for computers that is now known or to be developed in the future. The non-volatility of this storage means that the stored data is maintained regardless of whether power is being supplied to computer 102 and/or directly to persistent storage 120. Persistent storage 120 may be a read only memory (ROM), but typically at least a portion of the persistent storage allows writing of data, deletion of data, and re-writing of data. Some familiar forms of persistent storage include magnetic disks and solid-state storage devices. Operating system 130 may take any of several forms, such as various known proprietary operating systems or open source Portable Operating System Interface-type operating systems that employ a kernel.

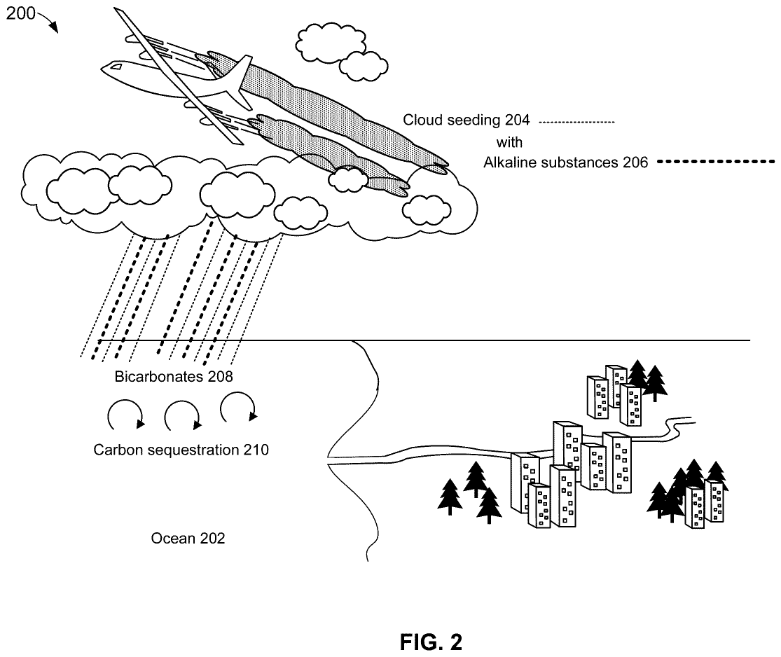

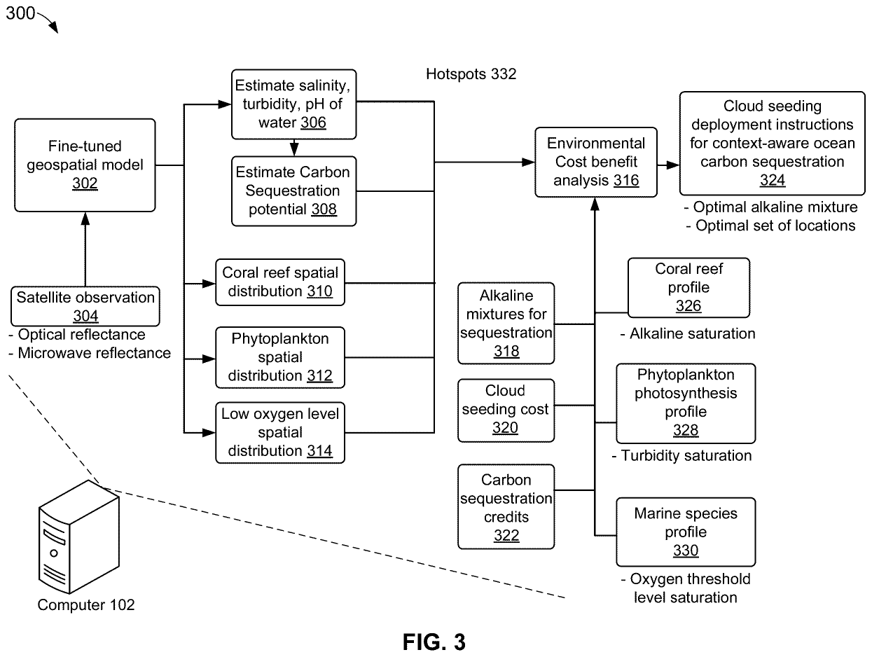

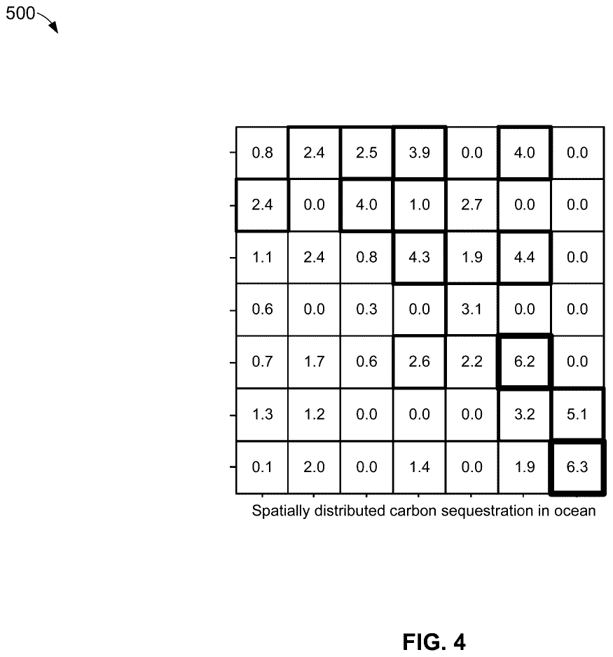

The code included in the carbon sequestration code 150 typically includes at least some of the computer code involved in performing one or more operations described herein, such as the operations of implementation 200 in FIG. 2, FIG. 3, and the processes described in FIGS. 4-6.

Peripheral device set 122 includes the set of peripheral devices of computer 102. Data communication connections between the peripheral devices and the other components of computer 102 may be implemented in various ways, such as Bluetooth® connections, Near-Field Communication (NFC) connections, connections made by cables (such as universal serial bus (USB) type cables), insertion-type connections (for example, secure digital (SD) card), connections made through local area communication networks and/or connections made through wide area networks such as the internet. In various embodiments, UI device set 132 may include components such as a display screen, speaker, microphone, wearable devices (such as goggles and smart watches), keyboard, mouse, printer, touchpad, game controllers, and/or haptic devices. Storage 134 is external storage, such as an external hard drive, or insertable storage, such as an SD card. Storage 134 may be persistent and/or volatile. In some embodiments, storage 134 may take the form of a quantum computing storage device for storing data in the form of qubits. In embodiments where computer 102 is required to have a large amount of storage (for example, where computer 102 locally stores and manages a large database), this storage may be provided by peripheral storage devices designed for storing very large amounts of data, such as a storage area network (SAN) that is shared by multiple, geographically distributed computers. IoT sensor set 136 is made up of sensors that can be used in Internet of Things applications. For example, one sensor may be a thermometer and another sensor may be a motion detector.

Network module 124 is the collection of computer software, hardware, and firmware that allows computer 102 to communicate with other computers through WAN 104. Network module 124 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 124 are performed on the same physical hardware device. In other embodiments (for example, embodiments that utilize software-defined networking (SDN)), the control functions and the forwarding functions of network module 124 are performed on physically separate devices, such that the control functions manage several different network hardware devices. Computer-readable program instructions for performing the inventive methods can typically be downloaded to computer 102 from an external computer or external storage device through a network adapter card or network interface included in network module 124.

WAN 104 is any wide area network (for example, the internet) capable of communicating computer data over non-local distances by any technology for communicating computer data, now known or to be developed in the future. In some embodiments, the WAN 104 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and/or edge servers.

EUD 106 is any computer system that is used and controlled by an end user (for example, a customer of an enterprise that operates computer 102), and may take any of the forms discussed above in connection with computer 102. EUD 106 typically receives helpful and useful data from the operations of computer 102. For example, in a hypothetical case where computer 102 is designed to provide a recommendation to an end user, this recommendation would typically be communicated from network module 124 of computer 102 through WAN 104 to EUD 106. In this way, EUD 106 can display, or otherwise present, the recommendation to an end user. In some embodiments, EUD 106 may be a client device, such as thin client, heavy client, mainframe computer, desktop computer and so on.

Remote server 108 is any computer system that serves at least some data and/or functionality to computer 102. Remote server 108 may be controlled and used by the same entity that operates computer 102. Remote server 108 represents the machine(s) that collect and store helpful and useful data for use by other computers, such as computer 102. For example, in a hypothetical case where computer 102 is designed and programmed to provide a recommendation based on historical data, this historical data may be provided to computer 102 from remote database 138 of remote server 108.

Public cloud 110 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 110 is performed by the computer hardware and/or software of cloud orchestration module 142. The computing resources provided by public cloud 110 are typically implemented by virtual computing environments that run on various computers making up the computers of host physical machine set 144, which is the universe of physical computers in and/or available to public cloud 110. The virtual computing environments (VCEs) typically take the form of virtual machines from virtual machine set 146 and/or containers from container set 148. These VCEs may be stored as images and may be transferred among and between the various physical machine hosts, either as images or after instantiation of the VCE. Cloud orchestration module 142 manages the transfer and storage of images, deploys new instantiations of VCEs, and manages active instantiations of VCE deployments. Gateway 140 is the collection of computer software, hardware, and firmware that allows public cloud 110 to communicate through WAN 104.

Some further explanation of VCEs will now be provided. VCEs can be stored as “images.” A new active instance of a VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 112 is similar to public cloud 110, except that the computing resources are only available for use by a single enterprise. While private cloud 112 is depicted as being in communication with WAN 104, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this example, public cloud 110 and private cloud 112 are both part of a larger hybrid cloud.

Cloud computing services and/or microservices (not separately shown in FIG. 1): private and public clouds 110 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to an “as a service” technology paradigm where content is being presented to an internal or external customer in the form of a cloud computing service. As-a-service offerings typically provide endpoints with which various customers interface. These endpoints are typically based on a set of application programming interfaces (APIs). One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with such tasks. Another category is Software-as-a-Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

In some implementations, a device (e.g., computer 102, computer system) may receive satellite observation data comprising optical and microwave spectral reflectance data for a body of water. The device may determine a salinity, a pH, and a water carbon sequestration potential of the body of water using the satellite observation data and a geospatial foundation model. The device may determine one or more hotspots of potential carbon sequestration in the body of water based on the salinity, the pH, the water carbon sequestration potential, a phytoplankton turbidity saturation profile, and a marine species oxygen threshold profile. The device may output, based on the salinity, the pH, and the water carbon sequestration potential, an indication of an optimal set of hotspots of the one or more hotspots and an optimal mixture of alkaline substances for cloud seeding for the optimal set of hotspots.

In some implementations, the device may receive satellite observation data of ocean water. The device may use the satellite observation data to fine-tune geospatial foundation models to infer salinity, pH, and water carbon sequestration potential of the ocean water. The device may determine one or more hotspots of potential carbon sequestration in the ocean water based on the salinity, the pH, and the water carbon sequestration potential. The device may perform an environmentally-aware cost-benefit analysis to determine an optimal set of hotspots of the one or more hotspots and an optimal mixture of alkaline substances for cloud seeding. The device may output an indication of the optimal set of hotspots and the optimal mixture of alkaline substances for cloud seeding.

In some implementations, the device may evaluate, by a geospatial modeling system, satellite-based optical and microwave spectral reflectance data to estimate water salinity, pH, and carbon sequestration potential in a given ocean region. The operations may include identifying, by the geospatial modeling system, hotspots of potential carbon sequestration in the ocean region based on the water salinity, the pH, and the carbon sequestration potential. The operations may include outputting, by the geospatial modeling system, an indication of an optimal set of hotspots of the hotspots and an optimal mixture of alkaline substances for cloud seeding based on enhancing ocean-based carbon sequestration and minimizing environmental harm.

FIG. 2 is a diagram of an example implementation 200 associated with cloud seeding for carbon sequestration. Cloud seeding is a form of weather modification that involves introducing substances into clouds to enhance precipitation, usually to induce rain or snow. The goal of cloud seeding is to stimulate the natural process of precipitation formation by providing additional nucleating agents that facilitate the growth of ice crystals or water droplets within clouds. There are several types of materials used for cloud seeding, depending on the type of cloud, the desired outcome, and the specific weather conditions. Silver iodide is the most commonly used material for cloud seeding. Silver iodide is a crystal-forming substance that can be introduced into clouds through aircraft-mounted generators or ground-based delivery systems. Silver iodide helps to form ice crystals, which can then grow into larger ice crystals and eventually become too heavy to remain suspended in the air, resulting in precipitation. Other materials may include carbamide, salt, or hygroscopic materials, such as sodium chloride or calcium chloride. Example implementation 200 shows cloud seeding in the air above the ocean 202 with a cloud seeding material 204.

The clouding seeding process may also use alkaline substances 206 in addition to the typical materials for cloud seeding 204. The alkaline substances may include, for example, calcium hydroxide or sodium hydroxide. These substances can help to convert carbon dioxide (sequestration 210) into dissolved bicarbonates 208, which can then be stored in the ocean 202. However, some water carbon sequestration may damage the habitats of living organisms, depending on the water conditions at different locations.

FIG. 3 is a diagram of an example implementation 300 associated with context-aware water carbon sequestration. A computer system (e.g., computer 102) may receive a satellite observation 304, such as an optical reflectance and a microwave reflectance for different regions of the ocean 202. The computer system may enter the satellite observation 304 into one or more geospatial models, such as a fine-tuned geospatial model 302. The geospatial models may output estimates 306 of water salinity, turbidity (cloudiness), and pH, and a carbon sequestration potential 308 of a given ocean region. The computer system may use the geospatial models to identify hotpots 332 of potential carbon sequestration in the regions based on the estimated salinity, the estimated turbidity, the estimated pH, and/or the carbon sequestration potential.

The computer system may take these estimates, a coral reef spatial distribution 310, a phytoplankton spatial distribution 312, and a low oxygen spatial distribution 314 for the hotpots and perform an environmental cost-benefit analysis for the regions to determine an optimal set of hotspots, from among the hotspots, and an optimal mixture of alkaline substances for cloud seeding for the optimal set of hotspots.

The spatial distributions mentioned may refer to the geographical patterns and concentrations of various oceanic features and phenomena that are relevant to carbon sequestration. These spatial distributions are estimated by the geospatial models using the satellite data and other inputs. The coral reef spatial distribution 310 may include geographical locations and concentrations of coral reefs in the ocean region. Coral reefs are important ecosystems that can be affected by changes in ocean chemistry and temperature, and their distribution is a factor in determining the optimal deployment strategy for alkaline substances. The phytoplankton spatial distribution 312 may include geographical locations and concentrations of phytoplankton in the ocean region. Phytoplankton are tiny plants that form the base of the ocean's food web and play a crucial role in the ocean's carbon cycle. Their distribution is a factor in determining the optimal deployment strategy for alkaline substances. The low oxygen zones spatial distribution 314 may include geographical locations and concentrations of low oxygen zones in the ocean region. Low oxygen zones, also known as “dead zones,” are areas where the oxygen levels are too low to support most forms of life. These zones can be affected by changes in ocean chemistry and temperature, and their distribution is a factor in determining the optimal deployment strategy for alkaline substances.

The computer system may use the geospatial models to estimate these spatial distributions using a combination of satellite data, oceanographic models, and other inputs. The estimated spatial distributions are then used to determine the optimal deployment strategy for the alkaline substances, taking into account the potential impacts on coral reefs, phytoplankton, and low oxygen zones.

The spatial distributions may be represented as maps or arrays of values, such as shown in FIG. 4, that represent the concentration or presence of each feature at different locations in the ocean region. These maps or arrays can be used to visualize and analyze the spatial patterns and relationships between the different features and to determine the optimal deployment strategy for the alkaline substances.

The computer system may output cloud seeding deployment instructions 324 for context-aware ocean carbon sequestration. The instructions may indicate an optimal alkaline mixture and an optimal set of locations, or hotspots, for the water carbon sequestration.

The computer system may use the cost-benefit analysis 316 to estimate the optimal amount of alkaline mixture for sequestration 318, a cloud seeding cost 320, carbon sequestration credits 322, and organism profiles, such as a combination of coral reef alkaline bicarbonate saturation profiles, phytoplankton turbidity saturation profiles, and marine species oxygen threshold profiles.

A coral reef alkaline bicarbonate saturation profile, such as coral reef profile 326, may describe the relationship between the concentration of alkaline substances and the saturation level of bicarbonates in coral reefs. Coral reefs have a limited capacity to absorb bicarbonates, and excessive levels can be detrimental to their health. The coral reef alkaline bicarbonate saturation profile helps to determine an optimal amount of alkaline substances that can be added to the ocean without harming the coral reefs.

A phytoplankton turbidity saturation profile, such as phytoplankton photosynthesis profile 328, may describe the relationship between the concentration of phytoplankton and the level of turbidity in the ocean. Phytoplankton can absorb certain wavelengths of light, which affects the ocean's turbidity. The phytoplankton turbidity saturation profile helps to determine an optimal amount of alkaline substances that can be added to the ocean without altering the phytoplankton's ability to absorb light.

A marine species oxygen threshold profile, such as marine species profile 330, may describe the relationship between the concentration of oxygen and the health of marine species. Marine species have different oxygen requirements, and excessive levels of alkaline substances can affect the ocean's oxygen levels. The marine species oxygen threshold profile helps to determine an optimal amount of alkaline substances that can be added to the ocean without harming marine species.

The profiles may be represented as mathematical functions or curves that describe the relationships between the different variables. The profiles can be derived from historical data, field observations, or laboratory experiments. For example, a coral reef alkaline bicarbonate saturation profile may be represented as a curve that shows the relationship between the concentration of alkaline substances and the bicarbonate saturation level in coral reefs. The curve may have a sigmoid shape, with the bicarbonate saturation level increasing rapidly at first and then leveling off as the concentration of alkaline substances increases.

In some implementations, the computer system may continuously monitor the effectiveness of the cloud seeding and provide feedback to adjust the deployment strategy as needed. For example, the system may use a combination of real-time data and machine learning models to monitor the body of water and determine the effectiveness of the optimal mixture. Ground truth data can be used to validate the accuracy of the models and make adjustments as needed. The computer system may use the optical and microwave spectral reflectance data to adjust the geospatial foundation models and improve the accuracy of the carbon sequestration potential estimates. The duration of effectiveness can be used to determine the optimal frequency of alkaline mixture deployment.

Additionally, or alternatively, estimating the carbon sequestration potential may include determining the optimal amount of alkaline mixture based on constraints of maximum alkaline bicarbonate saturation level of coral reefs. For example, the system may use historical data and machine learning models to determine the maximum alkaline bicarbonate saturation level of coral reefs and adjust the optimal amount of alkaline mixture accordingly.

Additionally, or alternatively, estimating the carbon sequestration potential may include determining the optimal amount of alkaline mixture based on constraints of maximum turbidity levels for phytoplankton photosynthetic activity. For example, the system may use real-time data and machine learning models to determine the maximum turbidity level for phytoplankton photosynthetic activity and adjust the optimal amount of alkaline mixture accordingly.

Additionally, or alternatively, estimating the carbon sequestration potential may include determining the optimal amount of alkaline mixture based on constraints of minimum oxygen levels for other marine species. For example, the system may use historical data and machine learning models to determine the minimum oxygen level for other marine species and adjust the optimal amount of alkaline mixture accordingly.

Additionally, or alternatively, determining the optimal set of hotspots may include performing an environmentally-aware cost-benefit analysis. For example, the environmentally-aware cost-benefit analysis may be used to evaluate the tradeoff between the additional gain from carbon credits for sequestration and the cost of deploying the optimal mixture. The alkaline mixture can be directly deployed into the ocean using a boat or other means, rather than relying on cloud seeding. Additionally, or alternatively, the alkaline mixture and cloud seeding mixture can be deployed separately, allowing for more precise control over the amount and location of each mixture to optimize the effectiveness of the alkaline mixture and the cloud seeding mixture.

An optimal mixture of alkaline substances for cloud seeding to enhance ocean carbon sequestration may include a combination of components, such as calcium hydroxide (Ca(OH)₂), sodium hydroxide (NaOH), magnesium hydroxide (Mg(OH)₂), or potassium hydroxide (KOH). An example of an optimal mixture may include 60% calcium hydroxide, 20% sodium hydroxide, 10% magnesium hydroxide, and 10% potassium hydroxide. This mixture would have a pH of around 12.5, which is highly alkaline and can help to promote the formation of bicarbonates in the ocean. The exact composition of the mixture may vary depending on the specific application and the desired outcome. The optimal mixture may depend on various factors such as the location, ocean chemistry, and weather conditions. The mixture may be designed to maximize the carbon sequestration while minimizing the potential harm to the marine ecosystem.

FIG. 4 is an example 400 of a spatial map. The map may include features such as hotspots (areas with exceptionally high carbon sequestration potential, marked with symbols or colors), or boundaries of marine protected areas, exclusive economic zones, or other areas of interest. The spatial map may provide a visual representation of the areas with high potential for carbon sequestration, allowing researchers and policymakers to identify priority areas for further study and potential intervention.

The spatial map may be, for example, for the spatial distribution of coral reefs, showing the location and density of coral reefs in the ocean region, with higher values indicating areas with more extensive or healthier coral reefs. Similarly, the spatial distribution of phytoplankton may be represented as a map showing the concentration of phytoplankton in the ocean region, with higher values indicating areas with more abundant phytoplankton. By analyzing and combining these spatial distributions, the geospatial modeling system can determine the optimal deployment strategy for the alkaline substances, taking into account the potential impacts on the ocean's ecosystems and the effectiveness of the carbon sequestration process. The deployment strategy may be a result of the environmental cost-benefit analysis 316, including an optimal deployment strategy for the alkaline substances, including the location, the amount, and the timing of deployment.

Given an n×n grid of identified spatial hotspots of potential water-based carbon sequestration, the optimal set of “j” may be selected such that sequestration will be coral-safe aware, by selecting the right mixture of alkaline without exceeding the coral reef alkaline saturation profile and minimizing the overall cost of deploying alkaline mixtures for cloud seeding based water carbon sequestration, considering the market for carbon credits. The computer system may use a cost function

$$J = Max {\sum }_{j = 1}^{N} {u}_{j} \{ {w}_{seq} \times {E}_{seq , j} ( alkj ) - {w}_{D} \times {C}_{j} ( alkj ) \} ,$$

where W_{seq}×E_{seq,j}(alkj) represents the increase in water carbon sequestration at pixel j for time t, and w_{D}×Cⱼ(alkj)} represents the cost of deploying cloud seeding at pixel j for time t.

The different elements of the constraints may include uⱼ for a binary decision variable (optimal identified hotspot pixel j); E_{seq,j} for an increase in water carbon sequestration at pixel j, which is a function of alkaline mixture alkj; Cⱼ (cost of deploying optimal alkaline mixture for cloud seeding at pixel j); w_{seq}, w_{D} (weighing coefficient for adding alkaline mixture for water carbon sequestration (carbon credit) and the expense for cloud seeding respectively); Bic_{seq,j}—bicarbonate formed in the water due to the application of alkaline mixture alkj; and Bic_{coral,j}—Coral reef alkaline bicarbonate saturation at pixel j.

The constraint equations may include Bic_{seq,j}(alkj)≤Bic_{coral,j}; turbidity_{seq,j}(alkj)≤turbidity_{phytoplankton,j}; and oxygen_{seq,j}(alkj)≤oxygen_{marine,j}. The constraints may ensure that the bicarbonate formed by the alkaline mixture does not exceed the coral reef alkaline bicarbonate saturation, the turbidity does not impact the photosynthetic activity of phytoplankton, and no low oxygen zones are created for a marine species.

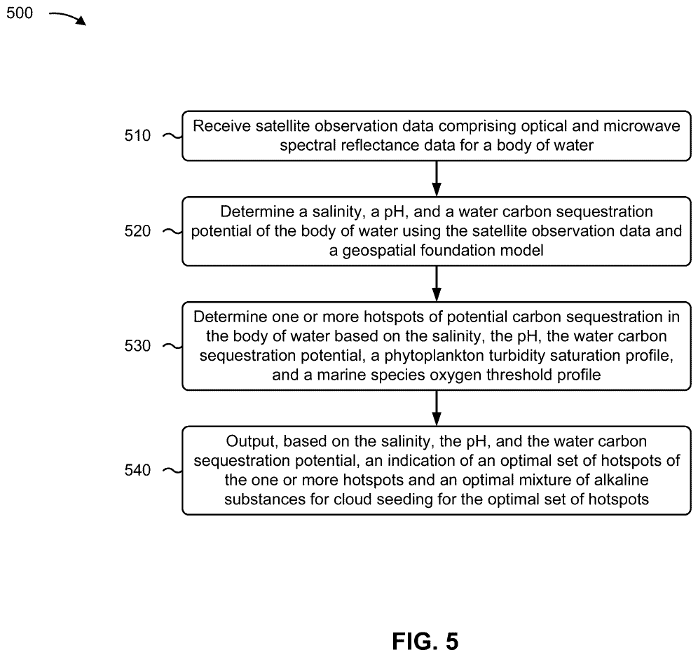

FIG. 5 is a flowchart of an example process 500 associated with context-aware cloud seeding for water carbon sequestration. One or more process blocks of FIG. 5 are performed by a computer system (e.g., computer 102) and/or by another device or a group of devices separate from or including the computer system.

As shown in FIG. 5, process 500 includes receiving satellite observation data comprising optical and microwave spectral reflectance data for a body of water (block 510). For example, the computer system may receive satellite observation data comprising optical and microwave spectral reflectance data for a body of water, as described above.

As further shown in FIG. 5, process 500 includes determining a salinity, a pH, and a water carbon sequestration potential of the body of water using the satellite observation data and a geospatial foundation model (block 520). For example, the computer system may determine a salinity, a pH, and a water carbon sequestration potential of the body of water using the satellite observation data and a geospatial foundation model, as described above.

As further shown in FIG. 5, process 500 includes determining one or more hotspots of potential carbon sequestration in the body of water based on the salinity, the pH, the water carbon sequestration potential, a phytoplankton turbidity saturation profile, and a marine species oxygen threshold profile (block 530). For example, the computer system may determine one or more hotspots of potential carbon sequestration in the body of water based on the salinity, the pH, the water carbon sequestration potential, a phytoplankton turbidity saturation profile, and a marine species oxygen threshold profile, as described above.

As further shown in FIG. 5, process 500 includes outputting, based on the salinity, the pH, and the water carbon sequestration potential, an indication of an optimal set of hotspots of the one or more hotspots and an optimal mixture of alkaline substances for cloud seeding for the optimal set of hotspots (block 540). For example, the computer system may output, based on the salinity, the pH, and the water carbon sequestration potential, an indication of an optimal set of hotspots of the one or more hotspots and an optimal mixture of alkaline substances for cloud seeding for the optimal set of hotspots, as described above.

Process 500 may include additional aspects, such as any single aspect or any combination of aspects described below and/or in connection with one or more other processes described elsewhere herein.

In a first aspect, the determining of the one or more hotspots comprises determining spatial distributions of coral reefs, phytoplankton, and low oxygen zones in the body of water.

In a second aspect, alone or in combination with the first aspect, process 500 includes determining the optimal set of hotspots and the optimal mixture, based on one or more constraints of identified sensitive zones and an environmentally-aware cost-benefit analysis.

In a third aspect, alone or in combination with one or more of the first and second aspects, the environmentally-aware cost-benefit analysis comprises evaluating a tradeoff between an additional gain from carbon credits for sequestration and a cost of deploying the optimal mixture.

In a fourth aspect, alone or in combination with one or more of the first through third aspects, the determining of the one or more hotspots comprises determining the one or more hotspots further based on a coral reef alkaline bicarbonate saturation profile and a phytoplankton turbidity saturation profile.

In a fifth aspect, alone or in combination with one or more of the first through fourth aspects, the geospatial foundation model includes a fine-tuned geospatial foundation model that is based on a plurality of machine learning models.

In a sixth aspect, alone or in combination with one or more of the first through fifth aspects, process 500 includes determining the optimal amount such that the optimal amount maximizes carbon sequestration without impacting a health of coral reefs, phytoplankton, and other marine species.

In a seventh aspect, alone or in combination with one or more of the first through sixth aspects, the determining of the optimal amount comprises determining the optimal amount based on constraints of maximum alkaline bicarbonate saturation level of the coral reefs, a maximum turbidity level for phytoplankton photosynthetic activity, or a minimum oxygen level for the other marine species.

In an eighth aspect, alone or in combination with one or more of the first through seventh aspects, process 500 includes providing instructions for deploying the optimal mixture to perform cloud seeding at the optimal set of hotspots.

In a ninth aspect, alone or in combination with one or more of the first through eighth aspects, the instructions are based on a result of an environmentally-aware cost-benefit analysis.

In a tenth aspect, alone or in combination with one or more of the first through ninth aspects, process 500 includes monitoring the body of water to determine an effectiveness of providing the optimal mixture to the optimal set of hotspots, and making adjustments to the optimal mixture and deployment strategy based on the monitoring.

Although FIG. 5 shows example blocks of process 500, in some implementations, process 500 includes additional blocks, fewer blocks, different blocks, or differently arranged blocks than those depicted in FIG. 5. Additionally, or alternatively, two or more of the blocks of process 500 may be performed in parallel.

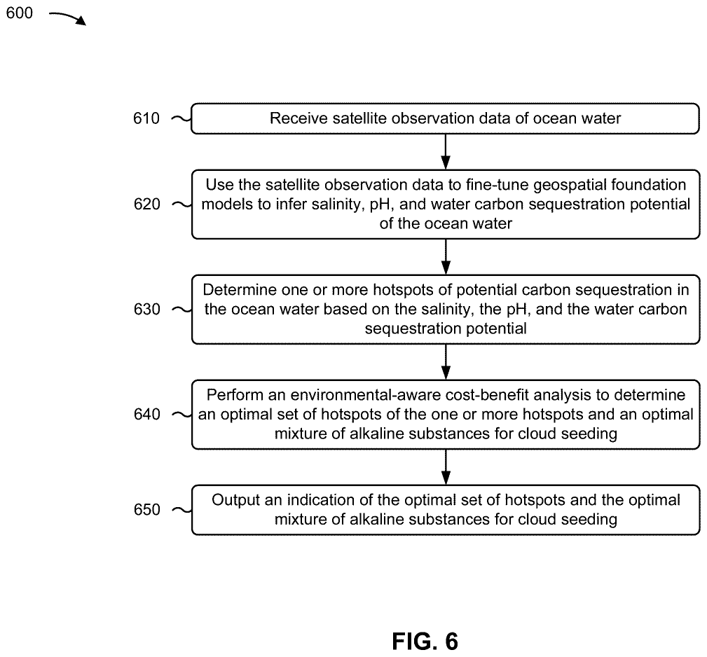

FIG. 6 is a flowchart of an example process 600 associated with context-aware cloud seeding for water carbon sequestration. One or more process blocks of FIG. 6 are performed by a computer system (e.g., computer 102) and/or by another device or a group of devices separate from or including the computer system.

As shown in FIG. 6, process 600 includes receiving satellite observation data of ocean water (block 610). For example, the computer system may receive satellite observation data of ocean water, as described above.

As further shown in FIG. 6, process 600 includes using the satellite observation data to fine-tune geospatial foundation models to infer salinity, pH, and water carbon sequestration potential of the ocean water (block 620). For example, the computer system may use the satellite observation data to fine-tune geospatial foundation models to infer salinity, pH, and water carbon sequestration potential of the ocean water, as described above.

As further shown in FIG. 6, process 600 includes determining one or more hotspots of potential carbon sequestration in the ocean water based on the salinity, the pH, and the water carbon sequestration potential (block 630). For example, the computer system may determine one or more hotspots of potential carbon sequestration in the ocean water based on the salinity, the pH, and the water carbon sequestration potential, as described above.

As further shown in FIG. 6, process 600 includes performing an environmentally-aware cost-benefit analysis to determine an optimal set of hotspots of the one or more hotspots and an optimal mixture of alkaline substances for cloud seeding (block 640). For example, the computer system may perform an environmentally-aware cost-benefit analysis to determine an optimal set of hotspots of the one or more hotspots and an optimal mixture of alkaline substances for cloud seeding, as described above.

As further shown in FIG. 6, process 600 includes outputting an indication of the optimal set of hotspots and the optimal mixture of alkaline substances for cloud seeding (block 650). For example, the computer system may output an indication of the optimal set of hotspots and the optimal mixture of alkaline substances for cloud seeding, as described above.

Process 600 may include additional aspects, such as any single aspect or any combination of aspects described below and/or in connection with one or more other processes described elsewhere herein.

In a first aspect, the satellite observation data includes optical and microwave spectral reflectance data, and the geospatial foundation models are fine-tuned using the optical and microwave spectral reflectance data.

In a second aspect, alone or in combination with the first aspect, the environmentally-aware cost-benefit analysis considers constraints of identified sensitive zones, including coral reefs, phytoplankton, and low oxygen zones.

In a third aspect, alone or in combination with one or more of the first and second aspects, the operations further comprise using the optimal set of hotspots and the optimal mixture of alkaline substances for cloud seeding to provide instructions for deploying alkaline mixtures to the ocean water for carbon sequestration.

In a fourth aspect, alone or in combination with one or more of the first through third aspects, the operations further comprise recursively performing the environmentally-aware cost-benefit analysis to optimize deployment of the optimal mixture for cloud seeding.

Although FIG. 6 shows example blocks of process 600, in some implementations, process 600 includes additional blocks, fewer blocks, different blocks, or differently arranged blocks than those depicted in FIG. 6. Additionally, or alternatively, two or more of the blocks of process 600 may be performed in parallel.

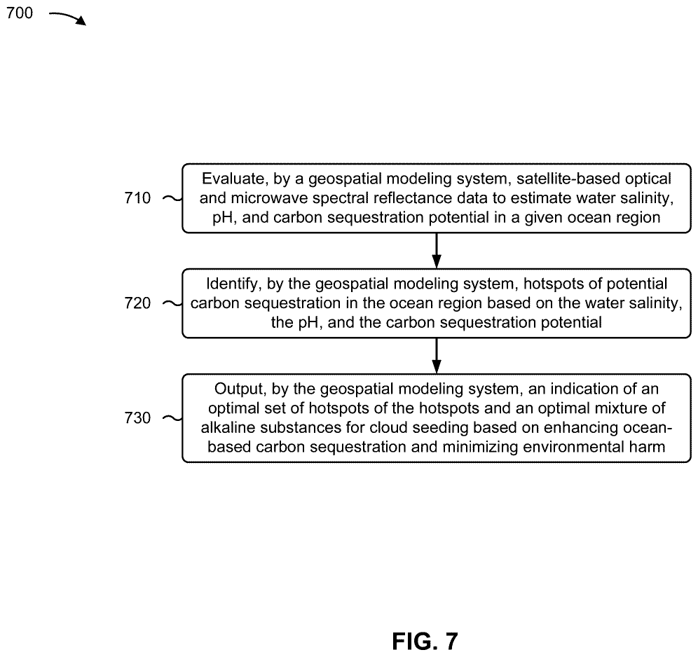

FIG. 7 is a flowchart of an example process 700 associated with context-aware cloud seeding for water carbon sequestration. One or more process blocks of FIG. 7 are performed by a computer system (e.g., computer 102) and/or by another device or a group of devices separate from or including the computer system.

As shown in FIG. 7, process 700 includes evaluating satellite-based optical and microwave spectral reflectance data to estimate water salinity, pH, and carbon sequestration potential in a given ocean region (block 710). For example, the computer system may evaluate satellite-based optical and microwave spectral reflectance data to estimate water salinity, pH, and carbon sequestration potential in a given ocean region, as described above.

As further shown in FIG. 7, process 700 includes identifying hotspots of potential carbon sequestration in the ocean region based on the water salinity, the pH, and the carbon sequestration potential (block 720). For example, the computer system may identify hotspots of potential carbon sequestration in the ocean region based on the water salinity, the pH, and the carbon sequestration potential, as described above.

As further shown in FIG. 7, process 700 includes outputting an indication of an optimal set of hotspots of the hotspots and an optimal mixture of alkaline substances for cloud seeding based on enhancing ocean-based carbon sequestration and minimizing environmental harm (block 730). For example, the computer system may output an indication of an optimal set of hotspots of the hotspots and an optimal mixture of alkaline substances for cloud seeding based on enhancing ocean-based carbon sequestration and minimizing environmental harm, as described above.

Process 700 may include additional aspects, such as any single aspect or any combination of aspects described below and/or in connection with one or more other processes described elsewhere herein.

In a first aspect, the operations further comprise estimating spatial distributions of coral reefs, phytoplankton, and low oxygen zones in the ocean region based on the satellite-based optical and microwave spectral reflectance data, and performing an environmental cost-benefit analysis to determine the optimal set of locations and the optimal mixture.

In a second aspect, alone or in combination with the first aspect, the environmental cost-benefit analysis considers a tradeoff between an additional gain from carbon credits for sequestration and a cost of deploying the optimal mixture at the optimal set of hotspots for cloud seeding.

In a third aspect, alone or in combination with one or more of the first and second aspects, the geospatial modeling system is associated with a combination of coral reef alkaline bicarbonate saturation profiles, phytoplankton turbidity saturation profiles, and marine species oxygen threshold profiles.

Although FIG. 7 shows example blocks of process 700, in some implementations, process 700 includes additional blocks, fewer blocks, different blocks, or differently arranged blocks than those depicted in FIG. 7. Additionally, or alternatively, two or more of the blocks of process 700 may be performed in parallel.

The foregoing disclosure provides illustration and description, but is not intended to be exhaustive or to limit the implementations to the precise forms disclosed. Modifications may be made in light of the above disclosure or may be acquired from practice of the implementations. For example, various aspects of this disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

A computer program product embodiment (“CPP embodiment” or “CPP”) is a term used in this disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, RAM, ROM, erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc), or any suitable combination of the foregoing. A computer-readable storage medium, as that term is used in this disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A computer-implemented method comprising:
receiving satellite observation data comprising optical and microwave spectral reflectance data for a body of water;
determining a salinity, a pH, and a water carbon sequestration potential of the body of water using the satellite observation data and a geospatial foundation model;
determining one or more hotspots of potential carbon sequestration in the body of water based on the salinity, the pH, the water carbon sequestration potential, a phytoplankton turbidity saturation profile, and a marine species oxygen threshold profile; and
outputting, based on the salinity, the pH, and the water carbon sequestration potential, an indication of an optimal set of hotspots of the one or more hotspots and an optimal mixture of alkaline substances for cloud seeding for the optimal set of hotspots.

2. The computer-implemented method of claim 1, wherein the determining of the one or more hotspots comprises:
determining spatial distributions of coral reefs, phytoplankton, and low oxygen zones in the body of water.

3. The computer-implemented method of claim 1, further comprising:
determining the optimal set of hotspots and the optimal mixture, based on one or more constraints of identified sensitive zones and an environmentally-aware cost-benefit analysis.

4. The computer-implemented method of claim 3, wherein the environmentally-aware cost-benefit analysis comprises evaluating a tradeoff between an additional gain from carbon credits for sequestration and a cost of deploying the optimal mixture.

5. The computer-implemented method of claim 1, wherein the determining of the one or more hotspots comprises determining the one or more hotspots further based on a coral reef alkaline bicarbonate saturation profile and a phytoplankton turbidity saturation profile.

6. The computer-implemented method of claim 1, wherein the geospatial foundation model includes a fine-tuned geospatial foundation model that is based on a plurality of machine learning models.

7. The computer-implemented method of claim 1, further comprising:
determining the optimal amount such that the optimal amount maximizes carbon sequestration without impacting a health of coral reefs, phytoplankton, and other marine species.

8. The computer-implemented method of claim 7, wherein the determining of the optimal amount comprises determining the optimal amount based on constraints of maximum alkaline bicarbonate saturation level of the coral reefs; a maximum turbidity level for phytoplankton photosynthetic activity; or a minimum oxygen level for the other marine species.

9. The computer-implemented method of claim 1, further comprising:
providing instructions for deploying the optimal mixture to perform cloud seeding at the optimal set of hotspots.

10. The computer-implemented method of claim 9, wherein the instructions are based on a result of an environmentally-aware cost-benefit analysis.

11. The computer-implemented method of claim 1, further comprising:
monitoring the body of water to determine an effectiveness of providing the optimal mixture to the optimal set of hotspots; and
making adjustments to the optimal mixture and deployment strategy based on the monitoring.

12. A computer system comprising:
a processor set;
one or more computer-readable storage media; and
program instructions stored on the one or more computer-readable storage media to cause the processor set to perform operations comprising:
receiving satellite observation data of ocean water;
using the satellite observation data to fine-tune geospatial foundation models to infer salinity, pH, and water carbon sequestration potential of the ocean water;
determining one or more hotspots of potential carbon sequestration in the ocean water based on the salinity, the pH, and the water carbon sequestration potential;
performing an environmentally-aware cost-benefit analysis to determine an optimal set of hotspots of the one or more hotspots and an optimal mixture of alkaline substances for cloud seeding; and
outputting an indication of the optimal set of hotspots and the optimal mixture of alkaline substances for cloud seeding.

13. The computer system of claim 12, wherein the satellite observation data includes optical and microwave spectral reflectance data, and the geospatial foundation models are fine-tuned using the optical and microwave spectral reflectance data.

14. The computer system of claim 12, wherein the environmentally-aware cost-benefit analysis considers constraints of identified sensitive zones, including coral reefs, phytoplankton, and low oxygen zones.

15. The computer system of claim 12, wherein the operations further comprise:
using the optimal set of hotspots and the optimal mixture of alkaline substances for cloud seeding to provide instructions for deploying alkaline mixtures to the ocean water for carbon sequestration.

16. The computer system of claim 12, wherein the operations further comprise:
recursively performing the environmentally-aware cost-benefit analysis to optimize deployment of the optimal mixture for cloud seeding.

17. A computer program product comprising:
one or more computer-readable storage media; and
program instructions stored on the one or more computer-readable storage media to perform operations comprising:
evaluating, by a geospatial modeling system, satellite-based optical and microwave spectral reflectance data to estimate water salinity, pH, and carbon sequestration potential in a given ocean region;
identifying, by the geospatial modeling system, hotspots of potential carbon sequestration in the ocean region based on the water salinity, the pH, and the carbon sequestration potential; and
outputting, by the geospatial modeling system, an indication of an optimal set of hotspots of the hotspots and an optimal mixture of alkaline substances for cloud seeding based on enhancing ocean-based carbon sequestration and minimizing environmental harm.

18. The computer program product of claim 17, wherein the operations further comprise:
estimating, by the geospatial modeling system, spatial distributions of coral reefs, phytoplankton, and low oxygen zones in the ocean region based on the satellite-based optical and microwave spectral reflectance data; and
performing, by the geospatial modeling system, an environmental cost-benefit analysis to determine the optimal set of locations and the optimal mixture.

19. The computer program product of claim 18, wherein the environmental cost-benefit analysis considers a tradeoff between an additional gain from carbon credits for sequestration and a cost of deploying the optimal mixture at the optimal set of hotspots for cloud seeding.

20. The computer program product of claim 17, wherein the geospatial modeling system is associated with a combination of coral reef alkaline bicarbonate saturation profiles, phytoplankton turbidity saturation profiles, and marine species oxygen threshold profiles.
